(19) United States
(12) Patent Application Publication
(10) Pub. No.: US 2018/0358129 A1
(43) Pub. Date: Dec. 13, 2018

(54) SYSTEM AND METHOD FOR EDUCATING A USER ABOUT A CONDITION OF INTEREST

(71) Applicant: KONINKLIJKE PHILIPS N.V., EINDHOVEN (NL)

(72) Inventors: Lukas Stefan GORZELNIAK, Aachen (DE); Jennifer CAFFAREL, Eindhoven (NL); Jarno Mikael RIISTAMA, Waalre (NL); Wilhelmus Johannes Joseph STUT, Eindhoven (NL); Mirela Alina WEFFERS-ALBU, Boukoul (NL)

(21) Appl. No.: 15/778,408

(22) PCT Filed: Nov. 23, 2016
(86) PCT No.: PCT/EP2016/078537

§ 371 Date: May 23, 2018

(30) Foreign Application Priority Data
Nov. 26, 2015 (EP) 15196541.5

(51) Int. Cl. G16H 50/70; G16H 50/50; G16H 70/00; G16H 80/00

(52) U.S. Cl. CPC G16H 50/70; G16H 80/00; G16H 70/00; G16H 50/50

(57) ABSTRACT

There is provided a method of operating a system to educate a user of the system about a condition of interest, the method comprising: receiving information that identifies a condition of interest; obtaining peer data from one or more databases, the one or more databases comprising health data about a plurality of people, the people each having one or more primary conditions, the health data including data about the one or more primary conditions and any secondary condition that affected the health or wellbeing of the people while the people were experiencing the one or more primary conditions; wherein the obtained peer data comprises the health data for people having a primary condition corresponding to the condition of interest; analyzing the peer data to determine a plurality of groups of people, wherein the people are grouped according to their secondary conditions; and generating a plurality of sets of visualization data based on the analysis of the peer data, each set of visualization data being generated based on the data for a respective determined group of people such that the visualization data can be used to visualize an evolution and/or outcome of the condition of interest and the secondary conditions affecting the people in the group to the user of the system.

### TECHNICAL FIELD OF THE INVENTION

The invention relates to a system and method for educating a user about a condition of interest.

### BACKGROUND OF THE INVENTION

Disease-specific education in how to manage a chronic condition (i.e. self-care) can reduce mortality and morbidity if medication is correctly used or symptoms and care instructions are well understood, for instance. However, health education is not routinely provided to patients with a chronic condition in the USA, and the percentage of patients who receive education can depend on the type of chronic condition.

Medical professionals lack the time to provide the required information to all patients or might communicate instructions only poorly (e.g. before discharge). This causes readmissions to the hospital within a short period of time due to low health literacy and insufficient knowledge of self-care medical instructions.

Patients on the other hand usually receive information on their condition very late in their patient journey, following an event (e.g. hospitalization) or during dedicated rehabilitation courses. Patients can also face instructions that are not easy to understand and might need to rely on information from peers to obtain the required knowledge. Information on how others cope with a condition, what treatment works for them, and what options exist are appreciated by patients.

Information needs also vary during different disease stages due to changes in symptoms, medication and/or functional capacity. Supporting educational tools are needed during the entire disease journey. Avatars such as virtual nurses and animated interactive online characters have been used to support self-care and have been shown to reduce readmissions and to promote behavior change.

There are a variety of educational resources is available for patients. They include: web-resources and booklets that provide general disease information without patient interaction; interactive educational games that focus on a single disease (and there are a limited amount of games available, so this is not for all patients or conditions); educational tools and apps are disconnected from professionals (e.g. no feedback is provided to the doctors treating the patient with recommendations on specific educational topics); and avatars are currently user-selected or represent health professionals or their functions;

### SUMMARY OF THE INVENTION

Of the education systems currently available, none take the patient's full condition(s) into account (e.g. multimorbidity and/or disease stages); none provide knowledge on current as well as future events, medication needs, symptoms etc.; and the disease experience of peers and psychological challenges are not integrated or considered.

In particular, ‘virtual nurse’ avatar systems are currently used to only support the discharge process (e.g. it asks about medication regimens and follow-up visits and assesses patients' understanding of medical instructions); it educates patients on diagnosis only during the hospital stay, i.e. there is no education at home; and the education does not include current or future symptoms (or medication aspects), or common comorbidities in the relevant patient group.

Therefore, there is a need for an improved system and method for educating a user (e.g. a subject/patient) about a condition of interest.

According to a first aspect, there is provided a method of operating a system to educate a user of the system about a condition of interest, the method comprising: receiving information that identifies a condition of interest; obtaining peer data from one or more databases, the one or more databases comprising health data about a plurality of people, the people each having one or more primary conditions, the health data including data about the one or more primary conditions and any secondary condition that affected the health or wellbeing of the people while the people were experiencing the one or more primary conditions; wherein the obtained peer data comprises the health data for people having a primary condition corresponding to the condition of interest; analyzing the peer data to determine a plurality of groups of people, wherein the people are grouped according to their secondary conditions; and generating a plurality of sets of visualization data based on the analysis of the peer data, each set of visualization data being generated based on the data for a respective determined group of people such that the visualization data can be used to visualize an evolution and/or outcome of the condition of interest and the secondary conditions affecting the people in the group to the user of the system.

According to a second aspect, there is provided a computer program product comprising a computer readable medium having computer readable code embodied therein, the computer readable code being configured such that, on execution by a computer or processor, the computer or processor is caused to perform the method described above.

According to a third aspect, there is provided a system for use in educating a user about a condition of interest, the system comprising a processing unit that is configured to: receive information that identifies a condition of interest; obtain peer data from one or more databases, the one or more databases comprising health data about a plurality of people, the people each having one or more primary conditions, the health data including data about the one or more primary conditions and any secondary condition that affected the health or wellbeing of the people while the people were experiencing the one or more primary conditions; wherein the obtained peer data comprises the health data for people having a primary condition corresponding to the condition of interest; analyze the peer data to determine a plurality of groups of people, wherein the people are grouped according to their secondary conditions; and generate a plurality of sets of visualization data based on the analysis of the peer data, each set of visualization data being generated based on the data for a respective determined group of people such that the visualization data can be used to visualize an evolution and/or outcome of the condition of interest and the secondary conditions affecting the people in the group to the user of the system.

Thus, the invention provides a way to educate patients/subjects and other system users on a condition of interest including complications and comorbidities that can arise as a result of that condition. This system provides several advantages, including a reduction in the education burden for health professionals (i.e. in having to educate patients); the provision of personalized disease education that is condition-based in the home environment; the provision of information about events (e.g. symptoms, treatment) common in similar patient groups; enables feedback (e.g. on knowledge gaps of patients) to health professionals; and ultimately lowers healthcare costs by avoiding or reducing readmissions through increased health literacy.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows a system according to an aspect of the invention;

FIG. 2 is a flow chart illustrating a method of operating a system about a condition of interest according to another aspect of the invention; and

FIG. 3 is a diagram illustrating an exemplary type of visual output for the system according to the invention.

### DETAILED DESCRIPTION OF THE PREFERRED EMBODIMENTS

As noted above, the education systems that are currently available do not take the patient's full condition(s) into account, do not provide knowledge on current as well as future events relating to the condition, and do not take into account disease experience of peers and psychological challenges. Therefore, the system and method described herein enable the education of a user about a condition of interest that addresses these limitations.

In particular, the invention provides that for a condition of interest (which may be a medical or health condition), health related data for peers that have the same or similar conditions is retrieved and analyzed to determine a plurality of peer groups, with the peers being grouped according to the way in which the condition of interest progressed over time, including common symptoms or outcomes, and/or other conditions or factors (i.e. other than the condition of interest) that affected the health or wellbeing of the peers while experiencing the condition of interest.

For example, where the condition of interest is heart failure, analysis of health related data for peers that have heart failure may determine five different peer groups, with a first peer group corresponding to those with well-managed heart failure, a second group corresponding to those with heart failure and diabetes, a third group corresponding to those with heart failure and chronic obstructive pulmonary disease (COPD), a fourth group with heart failure and COPD with disnatured (blue lips) and coughing with expectoration, and a fifth group corresponding to those with heart failure and pneumonia.

Once the plurality of peer groups have been determined, data for enabling the visualization of the evolution and/or outcome of the condition of interest according to each peer group is determined. This visualization data is used to display the evolution and/or outcome of the condition of interest to a user of the system so that the user can see how the condition may progress over time.

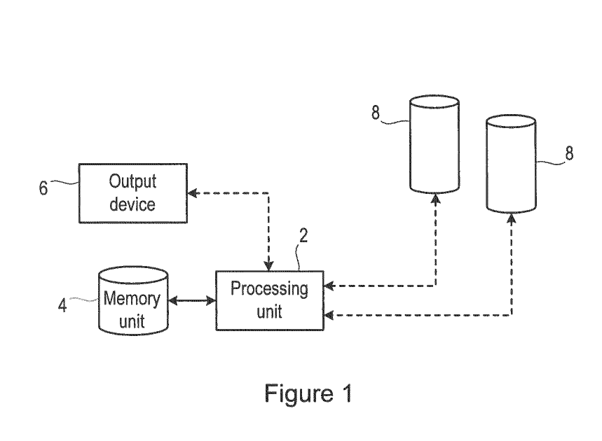

FIG. 1 shows a system according to an aspect of the invention. The system comprises a processing unit 2 that is connected to a memory unit 4. The processing unit 2 performs the main processing functions of the system as described in more detail below. The processing unit 4 can comprise one or more processors, control units, multi-core processors or processing modules. The processing unit 4 can be part of a smart phone, a general purpose computing device, such as a laptop, desktop computer, tablet, or a server.

The memory unit 4 can store program code that can be executed by the processing unit 4 to perform the method described herein. The memory unit 4 can also be used to store data that is collected or retrieved by the processing unit 2 during performance of the invention.

The system also comprises an output device 6 that is used to provide a visual output of the results of the processing by the processing unit 2. The output device 6 may be a display (e.g. a monitor or television), or a device 6 that comprises a display, e.g. a smart phone or a general purpose computing device, such as a laptop, desktop computer or tablet.

In some embodiments the processing unit 2 and output device 6 are part of the same device. However, in other embodiments, the processing unit 2 and output device 6 are implemented in separate devices. In this case, the processing unit 2 can be configured such that appropriate data representing the output of the processing according to the invention (e.g. the visualization data) can be provided to the output device 6 so that it can be displayed to the user of the output device 6. The processing unit 2 may communicate with the output device 6 via a wired or wireless connection, or via the Internet (for example where the processing unit 2 is a server and the output device 6 is a smart phone).

According to the invention, the processing unit 2 obtains or retrieves information from one or more databases 8, two of which are shown in FIG. 1. These databases 8 contain health and well-being related information for a (large) plurality of people. The databases 8 can be provided by external providers (for example healthcare providers (e.g. NHS UK: Health and Social Care Information Centre, PatientCrossroads™), health insurance companies, online intervention/engagement services (e.g. BigWhiteWall), online patient networks (e.g. PatientLikeMe), or registries etc.), and as such are not directly part of the system according to the invention. Instead, the processing unit 2 is able to request and retrieve specific information from the database(s) as required, for example via the Internet.

The health and well-being related information (health data) may include a variety of information representing the health, condition(s) and status of each person over the course of time, including, for example, the age, gender body mass index (BMI), primary diagnosis (also referred to as primary condition herein), disease stage, test results, comorbidities, etc. The conditions can include medical, mental and/or psycho-social conditions (which are otherwise referred to as conditions that affected the health and/or wellbeing of the person). Information that is particularly important for the operation of the invention includes any of: information on the progression of symptoms of the person's condition(s) over time (which could be provided in the form of test results, questionnaire responses, healthcare professional notes, etc.), the treatment provided to the person, the identification of any other condition or conditions that has or is affecting the health or wellbeing of the person while they have the primary condition (also referred to herein as comorbidities/secondary conditions), and the symptoms and/or treatment undertaken for that/those other condition(s). In more detail, the health data can include general and physical symptoms such as energy, fatigue, appetite, insomnia, pain, frailty (all of which can be obtained from a medical history obtained by a healthcare professional or through questionnaires), disease specific symptoms (e.g. COPD: chest tightness, cough, dyspnea, wheezing, etc.) (all of which can be acquired from disease-specific questionnaires, websites and/or other databases), additional symptoms such as balance problems, dry eyes, headaches, itching, etc. (all of which can be obtained from quality of life questionnaires and/or websites), laboratory test values such as blood tests, SpO2, other fluid/tissue samples, etc. (including normal ranges), mental disease and mood symptoms such as (external) stress, anxiety, depression, etc., and/or treatment options (which can be acquired from disease-specific guidelines, patient self-report etc.).

It will be appreciated that the health information in the database(s) 8 will cover a number of conditions, such that information can be extracted for people with a condition of interest. It will also be appreciated that a person's conditions indicated in the database(s) 8 may or may not be explicitly indicated as ‘primary’ and ‘secondary’/‘comorbidity, but instead may be presented as a set of conditions that the person is suffering from or experiencing/has suffered from or experienced. The data in the database(s) 8 may have been acquired from many different sources.

It will be appreciated that FIG. 1 only shows the components required to illustrate this aspect of the invention, and in a practical implementation the system may comprise additional components to those shown. For example, the system may comprise one or more user interface components that allow the subject or another user to interact with and control the system (and it will be appreciated that such a user interface can be provided at the output device 6 and/or at the processing unit 2. As an example, the one or more user interface components could comprise a switch, a button or other control means for enabling inputs to the system by a user.

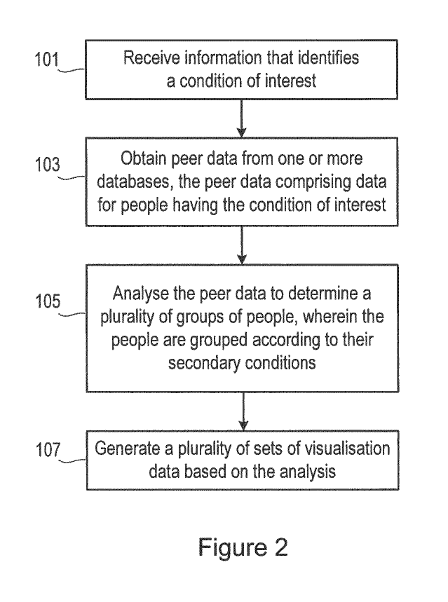

A general method of operating the system of FIG. 1 to educate a user of the system about a condition of interest is shown in FIG. 2. The system can be used by a patient or subject that has a condition of interest (e.g. a medical condition such as Chronic obstructive pulmonary disease (COPD)), and the output provided by the system can provide information to the patient or subject on ways in which their condition could evolve or develop over time, including the evolution or development of other conditions that might affect their health or wellbeing over time. Alternatively, the system can be used by someone wishing to learn about a condition of interest (for example a student or healthcare professional).

In a first step, step 101, the system receives information that identifies a condition of interest. This information can be provided by the user of the system, for example by using a user interface on the output device 6 or a user interface associated with the processing unit 2. The information that identifies a condition of interest could alternatively be provided by a healthcare professional when setting up the system for use by a patient (who is the user in this case).

The system then obtains peer data from one or more databases (step 103). As noted above, the one or more databases 8 comprises health data for a large number of people having different conditions (e.g. medical, mental and/or psycho-social conditions). The health data in the database(s) 8 can indicate the condition or conditions that the people have suffered from or experienced. Thus this health data can indicate a primary condition or conditions for the person, and any secondary conditions that affected the person's health or wellbeing while they were experiencing the primary condition(s). The peer data extracted from the one or more databases 8 is the health data/information for people having a primary condition corresponding to the condition of interest.

It will be appreciated that the information obtained from the databases 8 can be the health information for any or all people having the condition of interest as their primary diagnosis (i.e. the main condition that the person is suffering from/experienced), or for any or all people having the condition of interest, i.e. whether or not the condition of interest is the person's primary diagnosis.

Next, in step 105, the processing unit 2 analyses the peer data to determine a plurality of groups of people. This step aims to group people according to their secondary conditions that affected their health or wellbeing while they were experiencing the condition of interest.

In particular, the peer data will include health data for people that each had/have the condition of interest, and step 105 will comprise analyzing this health data to group the people covered by the health data according to the other condition(s) that have affected them while suffering from/experiencing the condition of interest. It will be appreciated that this grouping can be carried out by grouping other/secondary condition(s) that are the same as each other, or alternatively that are similar to each other (e.g. that affect the same part of the body, such as the respiratory system, circulatory system, etc., or that affect the person in the same way, such as raising blood pressure, increasing tiredness, etc., or that are considered to fall into a similar category of severity in terms of the effect on the people, e.g. mild, moderate, severe, etc., or that were treated using the same medication/treatment plan). Further details on the forming of the groups of people is provided below.

Once a plurality of groups of people have been determined, the processing unit 2 generates visualization data to enable a user of the system to visualize the different groups of people (step 107). As noted above, each group of people has been formed based on the same or similar secondary conditions that have affected their health or wellbeing while suffering from/experiencing the condition of interest, and each set of visualization data is generated based on some or all parts of the health data for the people in that group to enable the visualization of the evolution and/or outcome of the condition of interest, including the effects on the health or wellbeing of the people caused by the same or similar secondary conditions that affected the people in the group. For example the visualization data can be generated based on the symptoms of the people over time (i.e. the symptoms of the primary condition and the comorbidities/secondary conditions), the treatment(s)/medication(s) taken (for one or both of the primary condition and the comorbidities), the effect of the treatment(s)/medication(s), etc.

It will be appreciated that the visualization data can take any desired form. For example, in some embodiments the visualization data can be in a form that is suitable for display by a display device (e.g. the visualization data can be in the form of pixel values or another display format). In other embodiments, the visualization data can be in a form that requires some further processing before it can be displayed to the user of the system. For example in the preferred embodiments where the different groups of people are visualized using avatars, the visualization data generated in step 107 can correspond to data that enables an avatar-generation program/module/unit to generate an avatar that shows the characteristics of the appropriate group of people. This processing can be performed by the processing unit 2 or by a processing unit in another device (for example in the output device 6).

By grouping the peer data into a plurality of groups according to the secondary conditions that the people have experienced and generating visualization data for each of those groups, the system provides an advantage that the user of the system can be shown different evolutions/outcomes of a condition at any given time. A further advantage is that it has been found that a user that has a condition tends to perceive themselves as being in a different disease stage to the stage that they are actually in (and particularly they tend to consider themselves at a less severe disease stage), and the presentation of several different evolutions/outcomes enables them to understand their condition more clearly.

In some cases, since the group of peers having the condition of interest is divided into separate groups, the number of people in each group can be assessed to determine a likelihood of the respective evolution and/or outcome occurring. This likelihood can be based on the number of people in each group against the total number of people represented in the peer data obtained in step 103, and this likelihood can be presented to the user of the system along with the sets of visualization data.

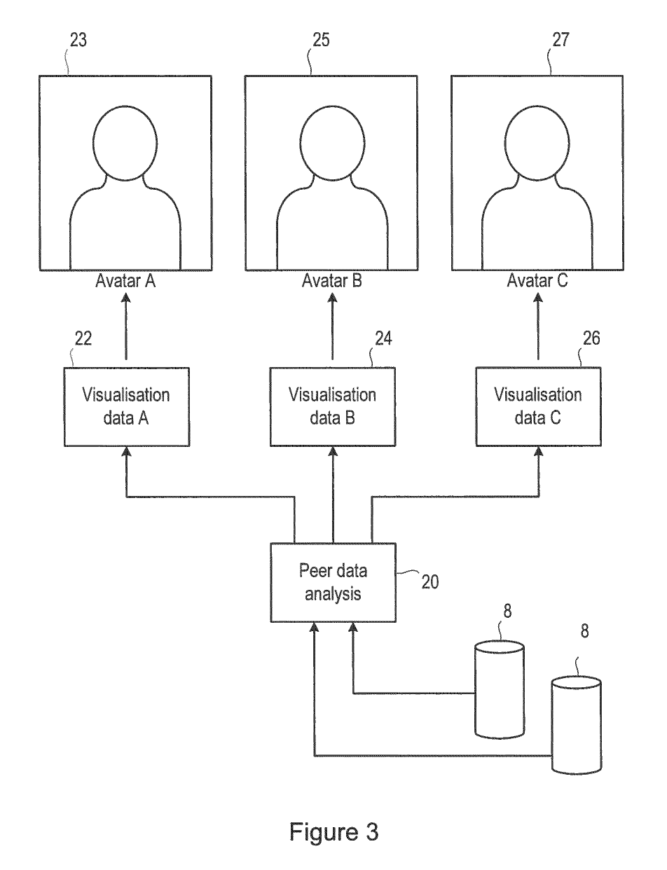

FIG. 3 is a diagram illustrating an exemplary type of visual output for the system according to the invention. FIG. 3 shows a peer data analysis module/unit 20 which performs the processing in step 105 of FIG. 2. It will be appreciated that peer data analysis module/unit 20 can be implemented by processing unit 2. The peer data analysis module/unit 20 obtains peer data from the one or more databases 8 and generates a set of visualization data for each identified group of people. In this example there are three sets of visualization data, visualization data A 22, visualization data B 24 and visualization data C 26, and each set of visualization data is used to generate a respective avatar on an output device 6. An avatar is a virtual representation of a person, and each avatar shows the evolution and/or outcome of the condition of interest and the effects of the relevant other condition(s) on the user. The avatar can be dynamic, e.g. in the sense that the visualization data can be used to generate an avatar that changes over time to show the evolution of the condition, or visualization data can be used to generate an avatar for a specific point or points in time (e.g. representing 1 month after diagnosis of the primary condition, 6 months after starting treatment A, etc.).

In the embodiment of FIG. 3, visualization data A 22 is used to generate Avatar A 23, visualization data B 24 is used to generate Avatar B 25, and visualization data C 26 is used to generate Avatar C 27. Thus, multiple avatars are presented to the user of the system to illustrate the different evolutions/outcomes of the condition of interest.

In some embodiments, the visualization data can be generated by taking into account the physical appearance of the user of the system so that the evolution and/or outcome of the condition of interest is presented using avatars that reflect the physical appearance of the user (e.g. the user can see what they might look like in the future as the condition progresses). Alternatively, the visualization data can be generated without reference to the physical appearance of the user, and the appearance of the avatar(s) can be based on some average of the obtained peer data or a ‘default’ avatar image.

In general, the appearance of an avatar can be determined by a number of appearance variables that relate to different aspects of the physical appearance of the avatar, and particularly aspects that can be affected by conditions of interest (e.g. height, face shape, body shape, weight, skin condition, skin color, hair quality/thinness, etc.). Thus, the visualization data can comprise values for these variables that are determined from the obtained peer data. The values can be determined by taking an average or weighted average of the peer data, for example. Alternatively the values can be based on a selection of the peer data, for example it may be desirable for the avatar to show extreme cases of the condition of interest and the specific comorbidity/ies associated with that peer group, in which case the values for the appearance variables can be determined from a subset of the peer data representing the peers with the most severe or extreme examples of the relevant symptoms.

An exemplary embodiment of the invention described above is set out below.

After a disease diagnosis or hospital admission, patient demographics and general information can be stored in an electronic health records (EHR) database. This database can be accessed by a system according to the invention (which is referred to as an education system (ES)) and/or information can be manually entered by a healthcare professional into the ES. Based on a condition of interest entered by the healthcare professional or other user, or a condition of interest identified in the information about the patient stored into the EHR database, the ES initiates a search to retrieve data of similar cases (i.e. people with the same conditions), including their symptoms, medication, treatments, outcomes, comorbidities (secondary conditions), etc.

The results of the search are stored in a knowledge database of the ES, e.g. memory unit 4 (i.e. cohorts based on real patient data).

The ES groups the results of the search according to the same or similar comorbidities.

In this exemplary use case, the ES groups the results of the search according to severity of their symptoms (e.g. mild, moderate and severe patients) and avatars are created based on the grouped information. Therefore these avatars will reflect mild, moderate and severe patients respectively.

In some embodiments, the ES can task the user of the ES to ‘take care’ of the avatars in order to educate the user about the ways in which the condition of interest can be treated and/or can progress over time. For example the user can be required to select treatment options (take medication A or B), make lifestyle choices (e.g. change amount of daily exercise, change diet, etc.) to see how these changes affect the avatars over time, and/or to find the best way of improving the symptoms exhibited by the avatars. It will be appreciated as well as the avatars illustrating the severity of the symptoms, a simple health tracker or status bar can be used to indicate the current severity of the avatar's symptoms to the user.

The ES will update the appearance of the avatars (i.e. update the visualization data used to generate the avatars) over time in accordance with the user's choices and selections based on the data stored in the knowledge database (that can indicate how the condition of specific patients changed when those same choices were made).

In addition, each avatar can experience certain situations in a “virtual pathway’ for the condition of interest, which can be determined from the information in the knowledge database. These situations can include exacerbations, the onset of depression, etc. The ES can provide information and education on these events to the user. The ES can prompt the user for input, while providing and informing the user about available options based on the data in the knowledge database.

For example, the user may see that the avatars doing well (e.g. with milder symptoms) in the morning. The user could be prompted for a choice (for all avatars or per avatar) such as taking a prescribed medicine or skipping the medication. If the medication is skipped the condition of the avatar will be affected accordingly based on data in the cohort (e.g. the condition of the avatar will deteriorate). The user may receive information from the ES to educate them why adhering to the medication regime is important.

Based on the choices made by the user of the system, the ES may identify gaps in the user's knowledge and report these gaps back to a healthcare professional.

An exemplary searching algorithm that can be used in step 103 is described in more detail below. In a first step, the ES performs a search of the EHR and any other suitable database using key search parameters (e.g. the identity of the condition of interest).

The ES retrieves patient profiles from the EHR having the condition of interest. If no patient profiles are found (or an insufficient number of patient profiles are found), the search can be broadened to identify patient profiles that have a similar condition to the condition of interest.

Optionally, the search can be repeated to identify patient profiles with less severe and more severe disease conditions (e.g. a different New York Heart Association (NYHA) Functional Classification class in heart failure patients).

In a further optional step, the ES retrieves possible pathways for the condition of interest from the EHR or other database to identify potential stages for the condition and/or evolution of the condition over time. If no pathway information is available, information in the patient profiles on the disease progression history is used instead.

An exemplary processing algorithm that can be used in step 105 to analyze the peer data to identify a plurality of groups of people based on the same or similar other conditions that affected their health or wellbeing while they were experiencing the condition of interest is described in more detail below.

In a first step, the ES extracts data from all profiles obtained from the database(s), e.g. symptoms, medication etc.

The ES then extracts treatment and medication information from disease guidelines. This data can be used to provide treatment options as well as information features.

In the next step, the ES determines the occurrence frequency of each variable (using descriptive statistics) to define ranks For example, it will be determined how often a variable, such as a “coughing” symptom, occurred in the patient group of interest, compared to other symptoms. Based on the occurrence frequency a ranking of this coughing symptom is determined. This is repeated for a number of other variables (e.g. other symptoms). Determining a frequency of occurrence of a variable using descriptive statistics will be known to those skilled in the art.

In the next step, information is connected by creating tuples such as (“Comorbidity”, Symptoms, Medication). This is necessary to e.g. link symptoms and treatments as different patient profiles might have been treated by different means for a similar condition (e.g. coughing). Therefore ranks will be assigned to determine which and how often certain symptoms occur within the comorbidity population, which and how often a therapy was used, etc. For example within a COPD population, a coughing symptom may be determined as one of the top 3 symptoms associated with this morbidity. As part of the same tuple, the common treatments for coughing are identified by the system and those with the top ranking (i.e. most frequent) are assigned. In this case, the rank information means the number of times these medications have been prescribed within the population group of interest with coughing symptoms. As an alternative, the medication ranking could indicate the most effective medication within the population group addressed. This can, for example, be determined by identifying the recurrence rate of exacerbation after the medication has been administered, as a measure of medication effectiveness based on the patient peer data. In other words, if patients having been prescribed medication X return for consultation with a healthcare professional with similar complaints after a certain period of time that is the longest period of time among all the patient groups treated with alternative medications, then based on this criterion medication X receives the highest ranking among all other medications.

Consider an example of patients with COPD and a coughing symptom. The system could find n=100 patients, where N=50 received medication A as treatment for coughing, N=30 received medication B as treatment for coughing; N=8 received medication C as treatment for coughing; N=6 receive homeopathy (which is denoted medication D); N=4 receive no treatment; and for N=2 no data is available.

A user of the system 2 is interested in the most common medications used for coughing. Based on the data above the system identifies that the top 3 (or top x) medications used for coughing are A, B and C. Now a tuple is created: (A, rank=1) (B, rank=2), (C, rank=3) which is provided to the system user.

If a user wishes to know how effective the medication is in the specific or a selected peer group, the system examines the outcomes of medications A, B and C on these patients. The possible outcomes can include: complaint-free time after prescription or the time between prescription and a follow-up visit for the same complaints/symptoms. The system can create a tuple of Medication, Rank, Result, e.g. (A, rank=1, effective 25%); (C, rank=3. Effective 75%).

It should also be noted that other factors can play a role, for example the level of patient adherence to use the prescribed medicine, or the effectiveness of a medication in a particular patient (for example a drug can be more effective in some people than others).

In a further step, examples of bad actions and consequences are recorded from the data in the cohorts (i.e. acquired past patient data from “similar” cases). For example, in a group of COPD patients who are not compliant with lifestyle recommendations and still smoke, there may be one group that was adherent to medication regimes and another group that was not. Based on data driven analysis between the groups, the consequences of this variable can be compared (e.g. hospital readmission or exacerbations).

An exemplary processing algorithm that can be used in step 107 to generate visualization data for avatars is described in more detail below. The user of the system can select the number of avatars that should be generated, or the number of avatars can be predefined in the system. Regardless of the number of avatars selected or predefined, the maximum number depends on the total number of groups identified in step 103.

Characteristics will be assigned to each avatar to reflect the characteristics of each cohort (group of people), based on descriptive statistics. The amount of variables assigned per avatar is determined by the average number of these variables among the retrieved information in the group. Each variable can be a comorbidity and/or a behavioral/emotional aspect, e.g. smoking habits.

Based on x retrieved profiles within one cohort of e.g. COPD GOLD III classified patients, an average of y symptoms is determined. This y is then used as the amount of assigned symptoms for the avatar. Within a specific sub-group (cohort) the amount of comorbidities between the retrieved profiles will vary (e.g. one retrieved patient profile could have 1 comorbidity, while another patient profile could have 20, for example). To accommodate for this variation the average of the number of comorbidities for all the retrieved profiles will determine the amount of the comorbidities of the avatar.

The y symptoms can consist of two sets of variables. The first set can consist of variables which are apparent in all or most of the patients of the obtained peer data, such as very common symptoms for the condition of interest (e.g. dyspnoea in COPD), treatment of this/these symptoms, medication, etc. The second set of variables can be selected from the remaining data, for example randomly or according to ranks (e.g. selecting those with a low rank), and can include information on more rare symptoms as well as information on the other condition(s) that the patients were suffering from/experiencing (e.g. symptoms such as depression). The avatar will have a number of appearance variables relating to its physical appearance (e.g. weight, skin condition, skin color, hair quality/thinness, etc.), and values for these variables will be determined from the peer patient profile data (for example by taking an average of peer patient profiles to determine the value for each appearance variable).

Where there is a conflict in the change in physical appearance caused by any of the y symptoms selected for the avatar, the change in physical appearance applied to the avatar corresponds to an average of the change in physical appearance experienced by those patients having those particular symptoms. For example, people with heart failure tend to increase in weight, while those with COPD tend to decrease in weight. For an avatar that is to represent heart failure and COPD symptoms, an average of the weight change for patient profiles with both heart failure and COPD can be determined, and that average used as an input to the avatar.

In some embodiments, it is possible to select a set of ‘extreme’ profiles for determining the avatar(s). For example a system user may be interested in seeing what the terminal stage of a disease (e.g. COPD) looks like. However, even in the last stages of COPD (e.g. GOLD IV with long-term oxygen therapy (LTOT)), there are differences between patients, as some have comorbidities, and others only suffer from COPD symptoms. The use of an average enables an avatar to be determined for this population group.

In a specific example of the above embodiments in which multiple sets of visualization data (e.g. for multiple avatars) are determined for COPD patients, one set can be determined from only the most common symptoms in COPD (e.g. based on averages and highest frequencies of occurrence), another set can be determined from a mix of common and rare symptoms and a third set can be determined from only the rare or very rare symptoms present in the patient population.

Therefore, the system and method described herein provide a user with education on diagnosis, symptoms, medication, treatment and/or psychological challenges associated with a condition of interest that is based on peer data for patients having the condition of interest.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method of operating a system to educate a user of the system about a condition of interest, the method comprising:
receiving information that identifies a condition of interest;
obtaining peer data from one or more databases, the one or more databases comprising health data about a plurality of people, the people each having one or more primary conditions, the health data including data about the one or more primary conditions and any secondary condition that affected the health or wellbeing of the people while the people were experiencing the one or more primary conditions; wherein the obtained peer data comprises the health data for people having a primary condition corresponding to the condition of interest;
analyzing the peer data to determine a plurality of groups of people, wherein the people are grouped according to their secondary conditions; and
generating a plurality of sets of visualization data based on the analysis of the peer data, each set of visualization data being generated based on the data for a respective determined group of people such that the visualization data can be used to visualize an evolution and/or outcome of the condition of interest and the secondary conditions affecting the people in the group to the user of the system.

2. A method as claimed in claim 1, wherein the method further comprises the step of:
using the plurality of sets of visualization data to display the evolution and/or outcome of the condition of interest and the secondary conditions affecting the people in each determined group to the user of the system.

3. A method as claimed in claim 1, wherein the method further comprises the step of:
sending the plurality of sets of visualization data to an output device for use by the output device in displaying the evolution and/or outcome of the condition of interest and the secondary conditions affecting the people in each determined group to the user.

4. A method as claimed in claim 1, wherein each set of visualization data comprises data for generating a respective avatar for each determined group of people such that the avatar visualizes the evolution and/or outcome of the condition of interest and the secondary conditions affecting the people in the group.

5. A method as claimed in claim 4, wherein each set of visualization data comprises values for a plurality of appearance variables.

6. A method as claimed in claim 1, wherein the step of analyzing the peer data to determine the plurality of groups of people comprises analyzing information on symptoms and/or behavioral and/or emotional aspects contained in the health data and grouping people based on combinations of symptoms and/or behavioral and/or emotional aspects for those people.

7. A method as claimed in claim 6, wherein the step of grouping people based on combinations of symptoms and/or behavioral and/or emotional aspects comprises:
determining the occurrence frequency of each symptom and/or behavioral and/or emotional aspect to define ranks;
determining the occurrence frequency of treatments and/or medications for the symptoms and/or behavioral and/or emotional aspects to define ranks; and
creating tuples to connect the symptoms and/or behavioral and/or emotional aspects to the treatments and/or medications based on the defined ranks.

8. A computer program product comprising a computer readable medium having computer readable code embodied therein, the computer readable code being configured such that, on execution by a computer or processor, the computer or processor is caused to perform the method of claim 1.

9. A system for use in educating a user about a condition of interest, the system comprising a processing unit that is configured to:
receive information that identifies a condition of interest;
obtain peer data from one or more databases, the one or more databases comprising health data about a plurality of people, the people each having one or more primary conditions, the health data including data about the one or more primary conditions and any secondary condition that affected the health or wellbeing of the people while the people were experiencing the one or more primary conditions; wherein the obtained peer data comprises the health data for people having a primary condition corresponding to the condition of interest;
analyze the peer data to determine a plurality of groups of people, wherein the people are grouped according to their secondary conditions; and
generate a plurality of sets of visualization data based on the analysis of the peer data, each set of visualization data being generated based on the data for a respective determined group of people such that the visualization data can be used to visualize an evolution and/or outcome of the condition of interest and the secondary conditions affecting the people in the group to the user of the system.

10. A system as claimed in claim 9, wherein the system is further configured to:
use the plurality of sets of visualization data to display the evolution and/or outcome of the condition of interest and the secondary conditions affecting the people in each determined group to the user of the system.

11. A system as claimed in claim 10, wherein the system further comprises an output device for displaying the evolution and/or outcome of the condition of interest and the secondary conditions affecting the people in each determined group using the visualization data.

12. A system as claimed in claim 9, wherein each set of visualization data comprises data for generating a respective avatar for each determined group of people such that the avatar visualizes the evolution and/or outcome of the condition of interest and the secondary conditions affecting the people in the group.

13. A system as claimed in claim 12, wherein each set of visualization data comprises values for a plurality of appearance variables.

14. A system as claimed in claim 9, wherein the processing unit is configured to analyze the peer data to determine the plurality of groups of people by analyzing information on symptoms and/or behavioral and/or emotional aspects contained in the health data and group people based on combinations of symptoms and/or behavioral and/or emotional aspects for those people.

15. A system as claimed in claim 14, wherein the processing unit is configured to group people based on combinations of symptoms and/or behavioral and/or emotional aspects by determining the occurrence frequency of each symptom and/or behavioral and/or emotional aspect to define ranks; determining the occurrence frequency of treatments and/or medications for the symptoms and/or behavioral and/or emotional aspects to define ranks; and creating tuples to connect the symptoms and/or behavioral and/or emotional aspects to the treatments and/or medications based on the defined ranks.
